(19) United States
(12) Patent Application Publication
(10) Pub. No.: US 2014/0276501 A1
(43) Pub. Date: Sep. 18, 2014

(54) Three-Dimensional Adhesive Patch

(71) Applicant: Hollister Incorporated, Libertyville, IL (US)

(72) Inventor: George J. Cisko, Spring Grove, IL (US)

(73) Assignee: Hollister Incorporated, Libertyville, IL (US)

(21) Appl. No.: 14/211,875

(22) Filed: Mar. 14, 2014

Related U.S. Application Data
(60) Provisional application No. 61/784,668, filed on Mar. 14, 2013.

(51) Int. Cl. A61F 5/449; A61F 5/443

(52) U.S. Cl. CPC A61F 5/449; A61F 5/443
USPC 604/344; 604/355

(57) ABSTRACT

An adhesive patch and its methods of application are provided. The adhesive patch comprises a curved base extending between an apex of the patch and a bottom of the patch, wherein the apex is located at a first end on a perimeter edge of the patch, the bottom is located at a second end on the perimeter edge, and the second end is opposite the first end. The patch further comprises a first side of the patch that extends from the curved base to the perimeter edge. The patch further comprises a hole in the curved base, wherein the curved base and the first side are configured to attach and conform to a three-dimensional region of a wearer's body, and wherein the hole is configured to align with an opening in the wearer's body to receive and facilitate the passage of liquids, solids, or gases from the wearer's body.

### RELATED APPLICATION DATA

This patent is related to and claims priority benefit of U.S. provisional application Ser. No. 61/784,668 filed Mar. 14, 2013 and entitled “Three-Dimensional Adhesive Patch.” The entire content of the prior filed provisional application is incorporated herein by reference.

### BACKGROUND

1. Field of the Disclosure

The present disclosure is generally directed to adhesive patches, and more particularly to a three-dimensional adhesive patch conformable to a three-dimensional (e.g., non-flat, curved, or complex curved region) region of the human body.

2. Description of Related Art

Fecal collector patches are known in the art. Known fecal collector patches are two-dimensional or flat patches. A two-dimensional fecal collector patch may be applied to the perianal region (or the area surrounding the anal opening) of a wearer's body. Known two-dimensional patches do not, however, optimally conform to the shape of the perianal region, which has an irregular, highly curved shape due to, for example, the curvature of the buttocks, the gluteal cleft, and the bottom of the gluteal cleft. As a result, gaps and channels may form between the two-dimensional patch and the perianal region, and the two-dimensional patch may be adhered to the perianal region in a stressed state. Gaps and channels are undesirable, as they may provide opportunities for leakage. These gaps and channels may be exacerbated, and/or new gaps and channels may form, when the two-dimensional patch is in the stressed state, as stressed portions of the patch tend to “relax.” Over time, these conditions may decrease the effectiveness of the patch, contribute to loss of wear time for the patch, cause discomfort for the wearer, and cause leakage.

### SUMMARY

The scope of the present invention is defined solely by the appended claims and is not affected to any degree by the statements within this summary. The present embodiments may obviate one or more of the drawbacks or limitations in the related art.

Adhesive patches and methods of application of the patches are provided. In one aspect, the adhesive patch comprises a curved base extending between an apex of the patch and a bottom of the patch, wherein the apex is located at a first end on a perimeter edge of the patch, the bottom is located at a second end on the perimeter edge, and the second end is opposite the first end. The patch further comprises a first side of the patch that extends from the curved base to the perimeter edge. The patch further comprises a hole in the curved base, wherein the curved base and the first side are configured to attach and conform to a three-dimensional region of a wearer's body, and wherein the hole is configured to align with an opening in the wearer's body to receive and facilitate the passage of liquids, solids, or gases from the wearer's body.

In another aspect, the adhesive patch comprises a curved base extending between an apex of the patch and a bottom of the patch, wherein the apex is located at a first end on a perimeter edge of the patch, the bottom is located at a second end on the perimeter edge, and the second end is opposite the first end. The patch further comprises a ridge within the curved base extending lengthwise between the apex and the bottom. The patch further comprises first and second sides of the patch that each extend from the curved base to the perimeter edge, wherein the first side is located on one side of the curved base, and the second side is located on an opposite side of the curved base. The patch further comprises a plurality of radial slits located on both the first side and the second side extending between the perimeter edge of the patch and the curved base, therein providing three or more petals on both the first side and the second side. The patch further comprises a hole in the ridge, wherein the curved base, the ridge, the first side, and the second side are configured to attach and conform to a three-dimensional region of a wearer's body, wherein the petals are configured to overlap when attached to the three-dimensional region of the wearer's body, and wherein the hole is configured to align with an opening in the wearer's body to receive and facilitate the passage of liquids, solids, or gases from the wearer's body.

In another aspect, a method of applying an adhesive patch to a wearer's body is provided. The method comprising providing an adhesive patch comprising: (a) a curved base extending between an apex of the patch and a bottom of the patch, wherein the apex is located at a first end on a perimeter edge of the patch, the bottom is located at a second end on the perimeter edge, and the second end is opposite the first end; (b) a ridge within the curved base extending lengthwise between the apex and the bottom; (c) first and second sides of the patch that extend from the curved base to the perimeter edge, wherein the first side is located on one side of the curved base, and the second side is located on an opposite side of the curved base; (d) a hole in the ridge; and (e) at least one release liner attached to a portion of the patch. The method further comprises folding the patch along the curved base, wherein the first side and the second side are moved closer together. The method further comprises removing the at least one release liner to expose an adhesive surface of the patch. The method further comprises applying the patch to a three-dimensional region of the wearer's body, wherein the curved base, the ridge, the first side, and the second side conform to the region of the wearer's body, and wherein the hole is aligned with an opening in the wearer's body to receive and facilitate the passage of liquids, solids, or gases from the wearer's body.

### BRIEF DESCRIPTION OF THE DRAWINGS

Objects, features, and advantages of the present embodiments will become apparent upon reading the following description in conjunction with the drawing figures, in which:

FIG. 1 depicts a perspective view of one embodiment of an adhesive patch.

FIG. 2 depicts a rear perspective view of the adhesive patch of FIG. 1.

FIG. 3 depicts the adhesive patch of FIG. 1 before the patch is applied to the perianal region of a wearer.

FIG. 4 depicts the adhesive patch of FIG. 1 with the curved base applied to the gluteal cleft of the perianal region of the wearer.

### DETAILED DESCRIPTION

The present embodiments are directed to three-dimensional or curved adhesive patches attachable to a three-dimensional region of the human body (e.g., a perianal or abdomen region). In certain embodiments, the three-dimensional adhesive patch is shaped and sized to optimally conform to the region of the human body when attached thereto. The contact area between the adhesive patch and the region may thus be increased. The stress caused by the attachment between the adhesive patch and the region may thus be reduced as well. In turn, the disclosed three-dimensional patch may be more effective, worn longer, be more comfortable for the wearer, reduce leakage, or a combination thereof.

The patch may be part of a bodily fluid management or collection system. In some examples, the patch may be part of a bowel management system or waste management system that includes a waste transport device, such as, for example, a fecal collection tube, bag, or a pouch, for a wearer. Examples are disclosed in, for example, U.S. Pat. No. 8,323,255, U.S. Pat. No. 8,075,540, U.S. Pat. No. 7,722,583, and U.S. Pat. No. 7,147,627, the contents of which are incorporated herein by reference in their entireties. The patch may be used to help securely couple a part of the waste transport device to the wearer's body. The patch may also help control odor and/or help prevent leakage in the waste management system. In other examples, the patch may be part of an ostomy or urostomy pouch system or may be used for wound exudate collection. The patch may be used for other purposes as well.

In certain embodiments, the curved adhesive patches includes one or more of the following features: (1) a curved base; (2) a perimeter edge of the patch, running around the circumference of the patch; (3) at least one side of the patch extending from the curved base to the perimeter edge of the patch; (4) an apex and a bottom of the patch, located on the perimeter edge of the patch at opposing ends; (5) a raised area or ridge running lengthwise along the curved base between the apex of the patch and the bottom of the patch; (6) a hole in the patch; and (7) at least one slit located on the at least one side of the patch, running between the perimeter edge of the patch and the curved base, wherein the at least one slit forms a plurality of petals (i.e., at least two petals) on the at least one side.

FIGS. 1 and 2 depict an adhesive patch 20 in accordance with the teachings of the present embodiments. The patch 20 is a three-dimensional or curved (i.e., not flat) adhesive patch. As will be described in greater detail below, the patch 20 may be attachable and conformable to a three-dimensional region of a wearer's body. In one example, the patch 20 is attachable and conformable to the perianal region of the wearer's body. In other examples, the patch 20 may be configured so as to be attachable and conformable to a different region, such as, for example, a portion of the abdomen.

As depicted in FIGS. 1 and 2, the curved adhesive patch 20 includes: a curved base 24; a patch perimeter edge 44; an apex 40 and a bottom 42 of the patch; a raised area or ridge 32 running lengthwise along the curved base 24 between the apex 40 and bottom 42 of the patch; a first side 28 and a second side 30, located on opposite sides of the curved base 24 and ridge 32; a hole 36 in the patch; and several slits 50 located on the first side 28 and the second side 30, running between the perimeter edge 44 and the curved base 32, wherein the slits forms a plurality of petals 48 on first side 28 and second side 30.

In certain embodiments, the patch includes a curved base and at least one side that extends outwardly (e.g., radially outward) from the curved base. In other embodiments, the patch includes a curved base and at least one side that extends outwardly from the ridge in the curved base. In the non-limiting example depicted by FIGS. 1 and 2, the patch 20 includes the curved base 24 and two opposing sides (e.g., a first side 28 and a second side 30) that extend outwardly from the curved base 24 (and the ridge 32 in the curved base 24). In other examples, the patch may include only one side, or more than two sides (e.g., three sides, four sides, etc.).

The curved base may be sized and shaped to approximate or conform to the natural size and shape (e.g., curvature or contour) of the desired region of the wearer's body. In certain embodiments, the curved base is sized and shaped to conform to the natural curvature of the perianal region. For example, the curved base may be sized and shaped to conform to the bottom of the gluteal cleft. In certain embodiments, the width of the curved base, or portions thereof, may be varied (e.g., increased) to provide better coverage and protection against the formation of sacral ulcers. In other embodiments, the shape of the curved base may be adjusted to conform to the natural curvature of the specific region (e.g., abdomen) of the wearer's body.

In certain embodiments, the patch may include a raised area or ridge located on the curved base of the patch. As shown in FIGS. 1 and 2, the curved base 24 of the patch 20 includes a raised area or ridge 32. The ridge 32 may provide extra thickness to a part of the three-dimensional region of the wearer, such as, for example, the base of the gluteal cleft. In certain embodiments, as depicted in FIGS. 1 and 2, the ridge 32 extends or runs through or along a central portion or region of the patch 20 between an apex 40 of the curved base 24 and a bottom 42 of the curved base 24. In other embodiments, the ridge may run along the perimeter edge of the patch between the apex and bottom of the patch. In some embodiments, the ridge may run along a length of the patch between the apex and bottom at some distance between the perimeter and central region of the patch. The ridge may extend or run along the entire length between the apex and bottom of the curved base. In other embodiments, the ridge may extend or run along a distance less than the entire length between the apex and bottom of the curved base.

The ridge may be of a uniform thickness, may be tapered, or a combination thereof (i.e., having segments of uniform thickness and other areas of a tapered thickness). The thickness of the ridge may be tapered in the lengthwise direction, in which case the thickness of the ridge is tapered between the apex of the curved base and the bottom of the curved base, and/or the widthwise direction, in which case the thickness of the ridge is tapered between a centerline of the ridge and the first and second sides.

In certain embodiments, the patch may include a hole or opening that extends through the patch. In some embodiments, the hole is an oval or circular shaped hole. The hole may be provided at any location of the patch. In certain embodiments, the hole is provided in the curved base or the ridge. In some embodiments, the hole is provided in a central location of the curved base or ridge. In other embodiments, the hole is provided in the curved base or ridge, at the apex or bottom, or proximal to the apex or bottom (i.e., closer to the apex or bottom than the center of the curved base/ridge).

The hole 36 in the depicted in FIGS. 1 and 2 as proximal or adjacent to the apex 40 of the curved base 24 and is located on the ridge 32 between the first side 28 and second side 30 of the patch 20. In embodiments where the patch 20 is attached to the perianal region (and, more particularly, the curved base 24 is attached to the bottom of the gluteal cleft), the hole 36 may thus be aligned with the anal opening of the wearer. The hole 36 may therefore be configured to receive and facilitate the passage of fecal matter. In other embodiments, such as, for example, when the patch 20 is attached to the abdomen, the hole 36 may be aligned with the stoma opening to receive and facilitate the passage of liquids, solids, and/or gases from the body. In any of these examples or in other examples, the location, size, and/or shape of the hole may vary from the hole 36 as depicted in FIGS. 1 and 2.

In certain embodiments, the at least one side that extends outward from the curved base has a perimeter edge. The width of a side may be uniform (i.e., the side has the same width throughout the length of the patch between the apex and the bottom). In other embodiments, the width of a side may be tapered throughout, or may have a combination of uniform and tapered sections. For example, a side may form a half circle or oval shape, where the width of the side (as measured from the ridge or curved base) is a minimum width at the apex, gradually increases to a maximum width near the center of the patch, and then gradually decreases toward a second minimum width at the bottom. The thickness of each individual side may likewise be uniform (i.e., the same thickness near or adjacent the ridge and at or along the perimeter edge edge). In other embodiments, the thickness of the side may be tapered (e.g., the thickness decreases in a radially outward direction), or the thickness may include a combination of uniform sections and tapered sections. For example, some portions of one or more of the sides may have a uniform thickness, while other portions may have a tapered thickness. In an embodiment where the patch includes opposing first and second sides, the first side may have the same width and thickness dimensions as the second side.

As shown in FIGS. 1 and 2, the patch 20 has a perimeter edge 44, and the first side 28 and the second side 30 both have a segment of the side that is tapered, wherein the width increases in the lengthwise direction from the apex 40 toward the bottom 42. In other embodiments, the side may have a segment that has a width that tapers in the opposite direction, such that the width increases from the bottom to the apex. In yet other examples, each side may have a uniform width or a partially uniform width and a partially tapered width (e.g., the bottom portion of the first side 28 or the second side 30 has a uniform width, while the top portion of the first side 28 or the second side 30 has a tapered width).

At least one side of the patch may be slit or cut to form two or more segments, sections, or petals. The at least one side of the patch may be radially slit and/or slit in some other direction (e.g., in an axial direction). In certain embodiments, at least one slit may be a radial slit that extends between the perimeter edge of the patch and the ridge, wherein the radial slit is in a direction that is orthogonal or perpendicular to the direction of the curved base or ridge. The radial slit may extend the entire length between the perimeter edge and the curved base or ridge, or some partial length concluding at a location short of the curved base or ridge. One or more of the petals formed from the slits may have the same shape and/or size. One or more adjacent petals may also overlap one another when the patch is applied to the desired region of the wearer. In any event, the petals may be moved relative to one another and may be attached to different portions of the desired region.

In the non-limiting example depicted in FIGS. 1 and 2, the first side 28 and the second side 30 each include three radial slits 50, such that four petals 48 are formed on each side. In other examples, more or less petals may be formed. As depicted, based on the location of the radial slits 50, the petals 48 have similar shapes and sizes. In certain embodiments, the petals 48 may facilitate a more flexible or adaptable shape and secure attachment between the patch 20 and the desired region of the body.

The patch may be made of or from one or more different materials or compositions. In some examples, the patch may be made of only one composition. In certain embodiments, the patch is made of a hydrocolloid material or barrier composition. In other embodiments, the patch may be made of rubber, paper, plastic, silicone, or a combination thereof. In some embodiments, the patch may be made of at least two different materials or compositions. For example, one portion of the patch (e.g., the curved base or the ridge) may be made of a first material, while a different portion of the patch (e.g., the sides or the ridge) may be made of a second, different material.

The exposed surface of the patch may include an adhesive surface. In some embodiments, at least a portion of an exposed surface of the patch is coated with an adhesive coating composition. For example, an exposed (body-facing) surface of the patch may be coated with an adhesive cross-linked composition or elastomeric barrier material. Non-limiting commercial examples of the elastomeric barrier materials include “Flextend,” or “SoftFlex” skin barriers, available from Hollister Incorporated (Libertyville, Ill.). In any event, in an embodiment where the patch has an adhesive surface, the patch is a self-adhesive patch and, thus, may be attached to and retained by the desired region of the wearer's body without separate taping or adhesive.

The patch may include at least one release liner attached to a portion of the patch, such as the sides (e.g., the petals) or ridge of the patch. The at least one release liner may be configured to protect the adhesive properties of the patch until application of the patch is desired. When application of the patch is desired, the one or more release liners may be removed and the patch may be conformed and applied to the desired region of the wearer's body. The one or more release liners may be simultaneously removed, or may be removed one at a time (e.g., as individual petals or portions of the patch are applied to the wearer) for easier placement and securement.

FIGS. 3 and 4 depict a non-limiting embodiment of how a patch 20 may be applied to the perianal region of the wearer's body. Provided the patch 20 depicted in FIGS. 1 and 2, a user (e.g., a medical professional or technician) may fold the first side 28 and second side 30 of the patch 20 about an axis defined by the curved base 24 or the ridge 32 of the patch 20. The first side 28 and second side 30 may fold inward toward one another until reaching the position shown in FIG. 3. In certain embodiments, after the patch has been folded, any release liners attached to patch 20, and in particular to the curved base 24 or the ridge 32, may be removed. In turn, as shown in FIG. 4, the curved base 24 and the ridge 32 may be applied between the buttocks 52 and adhered to the base of the gluteal cleft 54. In the position shown in FIG. 4, the curved base 24 of the patch 20 contacts the perineum and the hole 36 is aligned with the anus.

The user may then apply and affix the first side 28 and second side 30 to respective sides of the curved buttocks of the wearer within the gluteal fold. The user may remove the release liner attached to one of the petals 48 of the first side 28, and apply and affix the petal 48 to the appropriate side of the buttocks of the wearer. The remaining petals 48 of the first side 28 and the petals 48 of the second side 30 may be applied and affixed to the respective sides of the curved buttocks in the same way. Each petal 48 of each side is configured to adhere to the respective side of the buttocks with minimal resistance, such that the first side 28 and second side 30 are less likely to detach from the buttocks. One or more of the petals 48 may overlap one another as desired.

In other examples, the patch may be applied in a different manner and/or to a different region of the wearer's body. As another example, the patch may be applied to the abdomen of the wearer or to another region of the wearer's body. The curvature of the base and the number, shape, and arrangement of the petals may be adjusted in such an example to more optimally conform to the curvature of the abdomen or other region of the wearer.

Although certain adhesive patches have been described herein in accordance with the teachings of the present disclosure, the scope of coverage of this patent is not limited thereto. On the contrary, this patent covers all embodiments of the teachings of the disclosure that fairly fall within the scope of permissible equivalents.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

What is claimed is:

1. An adhesive patch comprising:
a curved base extending between an apex of the patch and a bottom of the patch, wherein the apex is located at a first end on a perimeter edge of the patch, the bottom is located at a second end on the perimeter edge, and the second end is opposite the first end;
a first side of the patch that extends from the curved base to the perimeter edge; and
a hole in the curved base,
wherein the curved base and the first side are configured to attach and conform to a three-dimensional region of a wearer's body, and
wherein the hole is configured to align with an opening in the wearer's body to receive and facilitate the passage of liquids, solids, or gases from the wearer's body.

2. The adhesive patch of claim 1, further comprising:
a ridge within the curved base extending lengthwise between the apex and the bottom, wherein the ridge is configured to conform to the three-dimensional region of the wearer's body.

3. The adhesive patch of claim 2, wherein the hole is located in the ridge.

4. The adhesive patch of claim 3, wherein the hole is located at or near the apex or the bottom of the patch.

5. The adhesive patch of claim 1, further comprising:
at least one radial slit located on the first side extending between the perimeter edge of the patch and the curved base, therein providing a plurality of petals on the first side,
wherein the petals are configured to overlap when attached to the three-dimensional region of the wearer's body.

6. The adhesive patch of claim 5, wherein the at least one radial slit comprises two or more slits on the first side, providing three or more petals on the first side.

7. The adhesive patch of claim 1, further comprising:
a second side of the patch that extends from the curved base to the perimeter edge, wherein the first side is located on one side of the curved base, and the second side is located on an opposite side of the curved base.

8. The adhesive patch of claim 7, wherein the first side and the second side have similar shapes.

9. The adhesive patch of claim 7, further comprising:
at least one radial slit located on both the first side and the second side extending between the perimeter edge of the patch and the curved base, therein providing a plurality of petals on both the first side and the second side,
wherein the petals are configured to overlap when attached to the three-dimensional region of the wearer's body.

10. The adhesive patch of claim 9, wherein the at least one radial slit comprises two or more slits on both the first side and the second side, providing three or more petals on both the first side and the second side.

11. The adhesive patch of claim 1, further comprising:
an adhesive coating on a surface of the patch.

12. The adhesive patch of claim 1, further comprising:
at least one release liner attached to a portion of the patch, wherein the at least one release liner is configured to protect the adhesive properties of the patch until application of the patch to the three-dimensional region of the wearer's body is desired.

13. The adhesive patch of claim 1, wherein the adhesive patch is part of a bodily fluid management or collection system.

14. The adhesive patch of claim 13, wherein the bodily fluid management is a bowel management system.

15. An adhesive patch comprising:
a curved base extending between an apex of the patch and a bottom of the patch, wherein the apex is located at a first end on a perimeter edge of the patch, the bottom is located at a second end on the perimeter edge, and the second end is opposite the first end;
a ridge within the curved base extending lengthwise between the apex and the bottom;
first and second sides of the patch that each extend from the curved base to the perimeter edge, wherein the first side is located on one side of the curved base, and the second side is located on an opposite side of the curved base;
a plurality of radial slits located on both the first side and the second side extending between the perimeter edge of the patch and the curved base, therein providing three or more petals on both the first side and the second side;
a hole in the ridge, wherein the curved base, the ridge, the first side, and the second side are configured to attach and conform to a three-dimensional region of a wearer's body, wherein the petals are configured to overlap when attached to the three-dimensional region of the wearer's body, and wherein the hole is configured to align with an opening in the wearer's body to receive and facilitate the passage of liquids, solids, or gases from the wearer's body.

16. The adhesive patch of claim 15, further comprising:
an adhesive coating on a surface of the patch; and
at least one release liner attached to a portion of the patch, wherein the at least one release liner is configured to protect the adhesive properties of the patch until application of the patch to the three-dimensional region of the wearer's body is desired.

17. A method of applying an adhesive patch to a wearer's body, the method comprising:
providing an adhesive patch comprising:
a) a curved base extending between an apex of the patch and a bottom of the patch, wherein the apex is located at a first end on a perimeter edge of the patch, the bottom is located at a second end on the perimeter edge, and the second end is opposite the first end;
b) a ridge within the curved base extending lengthwise between the apex and the bottom;
c) first and second sides of the patch that extend from the curved base to the perimeter edge, wherein the first side is located on one side of the curved base, and the second side is located on an opposite side of the curved base;
d) a hole in the ridge; and
e) at least one release liner attached to a portion of the patch;
folding the patch along the curved base, wherein the first side and the second side are moved closer together;
removing the at least one release liner to expose an adhesive surface of the patch;
applying the patch to a three-dimensional region of the wearer's body, wherein the curved base, the ridge, the first side, and the second side conform to the three-dimensional region of the wearer's body, and wherein the hole is aligned with an opening in the wearer's body to receive and facilitate the passage of liquids, solids, or gases from the wearer's body.

18. The method of claim 17, wherein the patch further comprises at least one radial slit located on both the first side and the second side, defining a plurality of radial slits, wherein each slit in the plurality of radial slits extends between the perimeter edge of the patch and the curved base, therein providing a plurality of petals on both the first side and the second side, and
wherein, during the applying, at least one petal in the plurality of petals overlaps at least one additional petal in the plurality of petals to conform the patch to the three-dimensional region of the wearer's body.

19. The method of claim 17, wherein the patch is applied to a bottom of a gluteal cleft and the hole is aligned with an anus of the wearer's body.

20. The method of claim 17, wherein the patch is applied to an abdomen and the hole is aligned with a stoma of the wearer's body.
